(19) United States
(12) Patent Application Publication
(10) Pub. No.: US 2024/0428478 A1
(43) Pub. Date: Dec. 26, 2024

(54) A SYSTEM AND METHOD FOR CONTOURING A MEDICAL IMAGE AND REVIEWING EXISTING CONTOURS

(71) Applicant: Boston Scientific Scimed, Inc., Maple Grove, MN (US)

(72) Inventors: Mark John GOODING, Oxford (GB); Padraig LOONEY, Oxford (GB); Djamal Boukerroui, Oxford (GB)

(21) Appl. No.: 18/699,293

(22) PCT Filed: Sep. 28, 2022
(86) PCT No.: PCT/EP2022/077010

§ 371 Date: Apr. 7, 2024

(30) Foreign Application Priority Data
Oct. 7, 2021 (GB) 2114397.9
May 23, 2022 (GB) 2207504.8

(51) Int. Cl. G06T 11/20; G06F 3/01; G06T 7/12; G06T 7/30

(52) U.S. Cl. CPC G06T 11/203; G06F 3/013; G06T 7/12; G06T 7/30; G06T 2200/24; G06T 2207/10081; G06T 2207/10088; G06T 2207/10104; G06T 2207/10108; G06T 2207/10132; G06T 2207/20081; G06T 2207/20104; G06T 2207/20128; G06T 2207/30004; G06T 2210/41

(57) ABSTRACT

A method and system for contouring a medical image in a contouring system is described. The method comprising the steps of: providing at least one medical image to be contoured by a user; determining that the user has initiated contouring of a structure on the medical image; the contouring system determining the structure that is being or will be contoured; in response to the determination of the structure the system displaying guidance for the contouring of the determined structure; contouring the determined structure in accordance with the displayed guidance. A method and system for reviewing contours on a contoured medical image is also described.

### FIELD OF INVENTION

This invention relates to the fields of medical imaging and medical image processing, in particular to the contouring of anatomy on a medical image or reviewing of pre-existing contours on a medical image and in particular in the field radiotherapy treatment planning.

### BACKGROUND OF INVENTION

In many scenarios, it is necessary for a clinician to outline anatomical structures on a medical image. For example, in radiotherapy treatment planning, organs, tumours and target volumes are typically delineated by a radiation oncologist on a medical image of the patient. The patient medical image is normally acquired with a Computed Tomography (CT) scan, although other imaging modalities, such as Magnetic Resonance Imaging (MRI), Positron Emission Tomography (PET) or Single Photon Emission Computed Tomography (SPECT), may be acquired. These medical images typically comprise a stack of 2D images of cross-sections of the patient, which together comprise a 3D volumetric image. When preparing for treatment, organs-at-risk (OARs) need to be delineated on a ‘planning’ image in order to spare healthy tissues. The planning software can subsequently calculate a treatment plan that maximises radiation dose to the target volume and tumour, while minimising dose to the surrounding healthy tissues. This process of delineation is known as contouring, a term that may be used to indicate the process of delineation in either 2D or 3D to define the boundary of an anatomical structure on the medical image. Similarly, the term auto-contouring may be used to indicate a contour on a medical image that is produced automatically.

Manual gold-standard contouring of a patient medical image by a human operator is time consuming, and subject to variability in delineating the anatomical structures on the medical image [1]. Such variability is due to intra-and inter-operator variation, and to variations between different institutions or departments. To reduce this variability, institutions and professional organisations produces contouring guidelines, such as [2], and atlases, such as [3]. The use of guidelines has been found to reduce variability between observers [4].

Guidelines take the form of a text description detailing how a contour of a structure should be drawn, while in this context, an atlas is a “gold-standard” set of contours drawn on an example patient image. Typically, guidelines and/or atlases are formed by consensus between experts.

While clinical teams are trained and educated in guidelines, over time variation can being to occur again where staff become complacent/forgetful [5], or guidelines get updated. Therefore, peer-review is often suggested as a way to check and remind staff of contouring guidelines. However, this process is time consuming and does not address (unintentionally) inter-institution variation.

Some technical solutions have been proposed to assist in adherence to guidelines. One approach is to use auto-contouring methods to produce contours from a system that should reproduce the guidelines. While auto-contouring has been shown to reduce variability between clinicians [6],knowledge of the guidelines is still required to correct any auto-contouring errors to adhere to those guidelines [5]. Another approach is to make those guidelines more easily accessible to the clinician while contouring (for example [7]). However, such an approach may interrupt the workflow of the clinician, requiring them to find the appropriate reference information as they work. Namely, a guideline may specify detail for a range of different structures, as the clinician contours one particular structure on a medical image, they would be required to find the relevant text within the guideline to check their contouring, even if presented with the correct set of guidelines. Furthermore, different institutions or geographic regions may define different protocols for treatment planning and may therefore have different contouring guidelines, and such solutions are not adapted for local deployment.
[1] Brouwer CL, Steenbakkers RJ, van den Heuvel E, Duppen JC, Navran A, Bijl HP, Chouvalova O, Burlage FR, Meertens H, Langendijk JA, van't Veld AA. 3D variation in delineation of head and neck organs at risk. Radiation Oncology. 2012 Dec; 7(1):1-0.
[2] Brouwer CL, Steenbakkers RJ, Bourhis J, Budach W, Grau C, Grégoire V, Van Herk M, Lec A, Maingon P, Nutting C, O'Sullivan B. CT-based delineation of organs at risk in the head and neck region: DAHANCA, EORTC, GORTEC, HKNPCSG, NCIC CTG, NCRI, NRG Oncology and TROG consensus guidelines. Radiotherapy and Oncology. 2015 Oct 1; 117(1):83-90.
[3] https://www.nrgoncology.org/About-Us/Center-for-Innovation-in-Radiation-Oncology/Head-and-Neck/Head-and-Neck-Atlases. Accessed 05/10/2021
[4] Sun KY, Hall WH, Mathai M, Dublin AB, Gupta V, Purdy JA, Chen AM. Validating the RTOG-endorsed brachial plexus contouring atlas: an evaluation of reproducibility among patients treated by intensity-modulated radiotherapy for head-and-neck cancer. International Journal of Radiation Oncology* Biology* Physics. 2012 Mar 1; 82(3):1060-4.
[5] Brouwer CL, Boukerroui D, Oliveira J, Looney P, Steenbakkers RJ, Langendijk JA, Both S, Gooding MJ. Assessment of manual adjustment performed in clinical practice following deep learning contouring for head and neck organs at risk in radiotherapy. Physics and imaging in radiation oncology. 2020 Oct 1; 16:54-60.
[6] Vinod SK, Min M, Jameson MG, Holloway LC. A review of interventions to reduce inter-observer variability in volume delineation in radiation oncology. Journal of medical imaging and radiation oncology. 2016 Jun; 60(3):393-406.
[7] https://anatom-e.com/verify/Accessed 05/10/2021

Thus, the following problem(s) has (have) been resolved, by the present invention; There is a need for a system and method to facilitate display of relevant guidance (guidelines and/or example atlas images) during contouring of one or more new contours on a medical image or during review or editing of one or more pre-existing contours on a medical image, related to the particular structure being contoured, to assist the clinical staff in outlining anatomical structures. Such a system and method is described and disclosed by the present invention.

According to the invention there is provided a method for contouring a medical image in a contouring system comprising the steps of: providing at least one medical image to be contoured by a user; determining that the user has initiated contouring of a structure on the medical image; the contouring system determining the structure that is being or will be contoured; in response to the determination of the structure the system displaying guidance for the contouring of the determined structure; contouring the determined structure in accordance with the displayed guidance.

In an alternative embodiment of the invention, there is also provided a method for reviewing contours on a contoured medical image comprising the steps of: providing at least one medical image annotated with one or more pre-existing contours; displaying the at least one medical image annotated with one or more pre-existing contours; determining that the user has initiated reviewing a pre-existing contour on the medical image; determine the structure associated with the pre-existing contour being reviewed by the user; in response to the determination of the structure displaying guidance for the contouring of the determined structure.

Preferably, the method further comprises the step of completing the review of the pre-existing contour or editing the pre-existing contour in accordance with the displayed guidance. Further preferably, completing the review of the pre-existing contour comprises providing feedback on the contour. In an embodiment of the invention, the feedback is provided as comments or annotation on the contour.

In a preferred embodiment, the method further comprising the step of outputting the user initiated contour or the reviewed contour associated with the image.

Preferably, the determination that the user has initiated contouring or reviewing is determined when the user starts to draw or edit a contour of the structure on the at least one medical image.

In a preferred embodiment of the invention the determination that a pre-existing contour is to be edited or reviewed is done using eye-tracking hardware and/or software to identify the location of the users gaze on the at least one medical image.

Preferably, the guidance for contouring is at least one of: clinical guidelines for contouring the determined structure or at least one atlas image showing at least one example contour for the determined structure.

Further preferably, the determination of the structure being reviewed or contoured is made by finding a mapping between the at least one medical image and the at least one atlas image. Preferably, the mapping is done using Deformable Image Registration. In a preferred embodiment of the invention the image registration comprises one or more of: deformable registration, affine registration or rigid registration

In an embodiment of the invention, the method further comprises using Machine Learning to predict the structure being contoured. Preferably, the determination of the structure being reviewed or contoured is made by comparison to contours produced using Machine Learning based auto-contouring. In an embodiment of the invention, the machine learning based auto-contouring uses one of: convolutional neural networks, random forest methods and support vector machines.

Preferably, the determination of the structure being reviewed or contoured on the medical image is done using eye-tracking hardware and/or software to identify the location of the users gaze on the at least one medical image.

In an embodiment of the invention, the contouring of the determined structure by the user is one of manual, semi-automatic or automatic contouring. Preferably, the contouring system determines the structure being contoured or reviewed using a contour name of the structure being contoured. Further preferably, the contour name is provided to a look-up table so the name is related to a standardised naming convention.

In an embodiment of the invention, the steps of the method are repeated so that multiple structures in the at least one medical image are contoured.

Preferably, the pre-existing contours displayed to the user are either manual or semi-automatic contours from a previous contouring session, or automatically produced contours.

In a preferred embodiment of the invention, the at least one medical image is one a CT scan, an MRI scan, a PET scan, or an ultrasound scan. Further preferably, the at least one medical image is one of a 2D image, a 3D image or a time series of medical images.

According to the invention there is also provided a system for contouring of at least one medical image comprising: a display for displaying at least one medical image to be contoured by a user; a processor for determining that the user has initiated contouring of a structure on the medical image; the processor determining the structure that is being contoured; in response to the determination of the structure the system displaying guidance to the user for the contouring of the determined structure; so that the determined structure can be contoured in accordance with the displayed guidance.

According to a further embodiment of the invention there is also provided system for reviewing or editing contours on a contoured medical image comprising: providing at least one medical image annotated with pre-existing contours; a display for displaying at least one medical image annotated with pre-existing contours; a processor for determining that a user has initiated reviewing a pre-existing on the image, the processor determining the structure of the pre-existing contour being reviewed; in response to the determination of the structure the system displaying guidance for the contouring of the determined structure; to allow completion of the contour review, or editing of the existing contour in accordance with the displayed guidance

### BRIEF DESCRIPTION OF THE DRAWINGS

Further details, aspects and embodiments of the invention will be described, by way of example only, with reference to the drawings. In the drawings, like reference numbers are used to identify like or functionally similar elements. Elements in the figures are illustrated for simplicity and clarity and have not necessarily been drawn to scale.

FIG. 1(a) is a flow chart showing the method according to an embodiment of the invention;

FIG. 1(b) is a flow chart showing the method according to an alternative embodiment of the invention;

FIG. 2(a) is a flow chart showing the method according to a further alternative embodiment of the invention;

FIG. 2(b) is a flow chart showing the method according to a further alternative embodiment of the invention;

FIG. 3 is an example user interface according to an embodiment of the invention;

FIG. 4 illustrates a simplified block diagram of an example of a medical imaging system.

### DETAILED DESCRIPTION

This invention is a system and method for enabling efficient contouring, or delineation, of structures on a medical image, or for the review of existing contours, along with the active display of relevant guidance information to assist the user during the contouring, or during review of existing contours. Preferably, the guidance information for the user is at least one of clinical guidelines for contouring a determined structure on a medical image or at least one atlas image showing at least one example contour of the determined structure on the medical image, generated according to the clinical contouring guidelines.

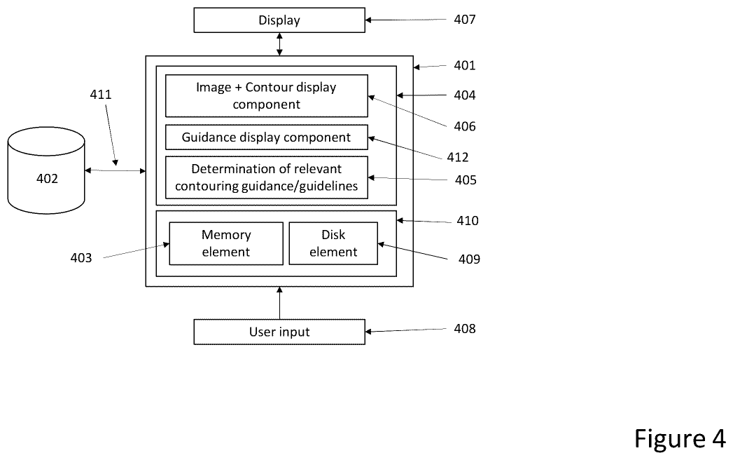

Referring now to FIG. 4, there is illustrated a simplified block diagram of an example of a medical imaging system 400 arranged to enable medical images to be displayed to a user, either for contouring or for review of existing contours. In a preferred embodiment of the invention the system comprises a display for displaying at least one medical image, where the medical image is to be contoured, or is already annotated with pre-existing contours to be reviewed. In the illustrated example, the medical imaging system 400 comprises one or more user terminals 401, for example comprising a workstation or the like, arranged to access medical images stored within, for example, a database 402 or other data storage apparatus. In an embodiment of the invention, these may provide at least one medical image annotated with pre-existing contours, or a medical image that is to be contoured. In the illustrated example, a single database 402 is illustrated.

However, it will be appreciated that the user terminal 401 may be arranged to access medical images from more than one data storage apparatus. Furthermore, in the illustrated example the database 402 is illustrated as being external to the user terminal 401. However, it will be appreciated that the user terminal 401 may equally be arranged to access medical images stored locally within a local storage module illustrated at 410 on one or more internal storage elements, such as the memory element illustrated at 403 or the disk element illustrated at 409.

In an embodiment of the invention for contouring a medical image, the workstation may comprise a processor for determining that the user has initiated contouring of a structure on the medical image; the processor determining the structure that is being contoured; in response to the determination of the structure the system displaying guidance to the user for the contouring of the determined structure; so that the determined structure can be contoured in accordance with the displayed guidance.

In an alternative embodiment of the invention, for reviewing pre-existing contours on a medical image the processor determines that a user has initiated reviewing a pre-existing contour on the image, the processor also determines the structure of the pre-existing contour being reviewed; in response to the determination of the structure the system displaying guidance for the contouring of the determined structure; to allow completion of the contour review, or editing of the existing contour in accordance with the displayed guidance.

The user terminal 401 further comprises one or more signal processing modules, such as the signal processing module illustrated generally at 404. The signal processing module(s) is/are arranged to executing computer program code, for example stored within local storage module 410. In the illustrated example, the signal processing module(s) 404 is/are arranged to execute computer program code comprising one or more of the automated components(s) illustrates in the example as; the component that automatically detects the relevant contouring guidelines or guidance for the structure being contoured 405. The signal processing module 404 in the illustrated example is further arranged to execute computer program code comprising one or more image display component(s) 406; The signal processing module 404 in the illustrated example is further arranged to execute computer program code comprising one or more guideline or guidance display component(s) 412; the guidance display component(s) 412 being arranged to the relevant portion(s) or example(s) of the guidance for contouring as determined by 405 in response the user interaction with the image and contour display 406, to a user, for example on a display screen 407 or the like.

The guidance selected by 405 and displayed by 412 may be loaded from a database 402, memory 403, or disk 409. The medical imaging system 400 may further comprise one or more user input devices, such as illustrated generally at 408, to enable a user to interact with computer program code etc. executing on the signal processing module(s) 404. After interaction the user instruct the system to save results back to local storage 410 or to a database 402, as indicated by the two-way directional arrow 411.

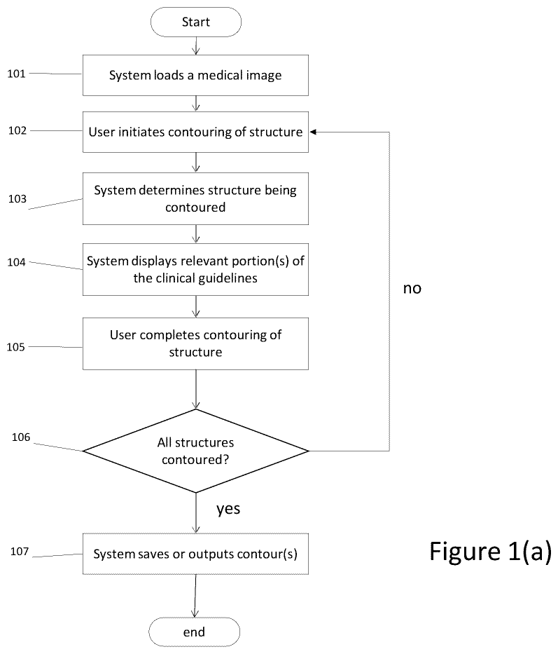

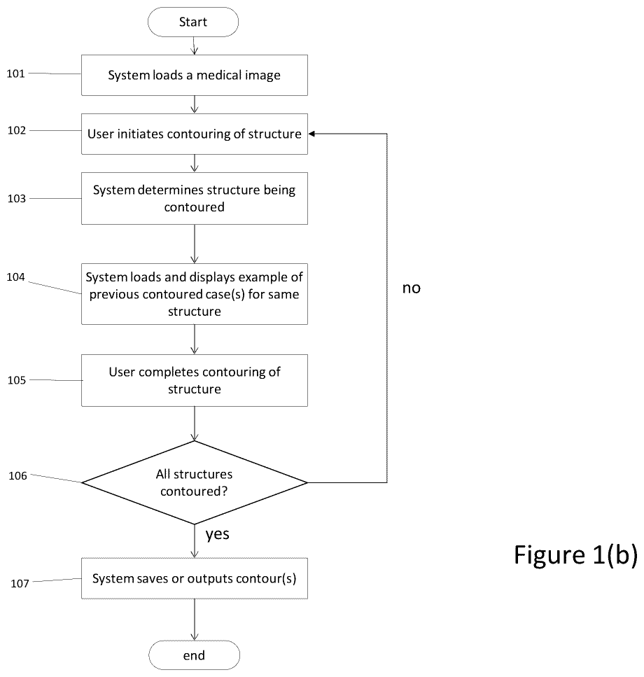

One embodiment of the invention is illustrated by FIG. 1(a). The system loads at least one a medical image to be contoured by a user (which may be 2D or 3D, or a time-series of medical images, where a time series is a series of 2D or 3D images taken in a single scanning session, where, for instance, each image may represent a different phase of a respiratory or cardiac cycle), at step 101. Preferably the medical image is a CT image, but other images such as MRI, PET, SPECT or Ultrasound images may also be possible. This loading process may have been user initiated or may have been automatic. The medical images may be provided from a database or from a file system or an external storage device, such as a disk. It is anticipated that such a system has normal viewing capabilities of the medical image visualisation tool, and the user may opt to change the view. At some point, after the medical images have been loaded it is determined that the user initiates the contouring process for the medical image, at step 102, using manual, semi-automatic or automatic contouring tools available in the system. Tools commonly provided by such systems include point-based polygon drawing tools, freehand pen-like tools and brush-like area-filling tools. Preferably, the determination that the user has initiated contouring or reviewing of a contour is determined when the user starts to draw or edit a contour of the structure on the at least one medical image. This may be done my tracking movement of the cursor on the display screen for example. In an alternative embodiment of the invention, the determination that a contour is being generated, edited or reviewed is done using eye-tracking hardware and/or software to identify the location of the users gaze on the at least one medical image. Once the contouring has been initiated the system then determines which anatomical structure on the medical image is being contoured or will be contoured by the user at step 103. Preferably, this determination of the structure on the medical image that is being contoured is done automatically by the system. Examples of methods for doing this are described in more detail below, but an example may be using the contour name (as provided by the user) for the contour currently being reviewed or generated, with a look-up table or database that is used to relate user specified structure names back to standard naming conventions (e.g. [8]) thus the structure that is being contoured is indicated by the user.

On determining which structure in the medical image is being contoured by the user, the system displays guidance information to the user about the contour for the structure being contoured. As described above, the guidance for the user is at least one of clinical guidelines for contouring a determined structure or at least one atlas image showing at least one example contour of the determined structure on the medical image, generated according to the clinical contouring guidelines. The structure can then be contoured in accordance with the displayed guidance. Preferably the guidance information is one or more of the relevant portions of guidelines to the user, at step 104, displayed to the user via the systems user interface. In a preferred embodiment of the invention the display of the guidance is an interactive display, based on the user behaviour as the contouring is performed. This interactivity reduces the time required to find the appropriate guidance or sections of the guidelines. This is in contrast to previous systems and methods for contouring where for example, an image to be contoured is shown in one screen, and general guidelines are displayed on another screen, where the user will have to manually scroll through the displayed guidelines to arrive at the appropriate guideline for the structure currently being contoured.

In some embodiments of the invention the guidance information may be an atlas. This is illustrated in FIG. 1(b) with items corresponding numbers to FIG. 1(a) being equivalent, the display of guidelines 104 is replaced by a graphical display of one or more example cases and contours (atlases) for the same structure.

For both of the embodiments in FIGS. 1(a) and 1(b), the user can then review the guidance (one or more of guidelines and/or atlases) while continuing to contour the structure on the medical image, to assist the user with the contouring process. The user then completes contouring of the structure, at step 105. The process of contouring structures (steps 102-105) may be repeated for multiple different structures on the medical image, so multiple structures in the at least one medical image are contoured, until the user is satisfied at step 106. If all the structures have been contoured then the workflow proceeds to step 107. If there are some structures that still have to be contoured then the workflow returns to step 102. In this manner, it is possible that the system is able to update the guidance for each iteration, and will only display the guidance for the contour that is currently being generated, rather than the guidance for a previous contour that has since been finalised. The system will then save, store, or output one or more of the delineations created by the user for further use in the clinical workflow at step 107. The process may also be repeated for other medical images, so that a series of images may be contoured by a user in a single contouring session.

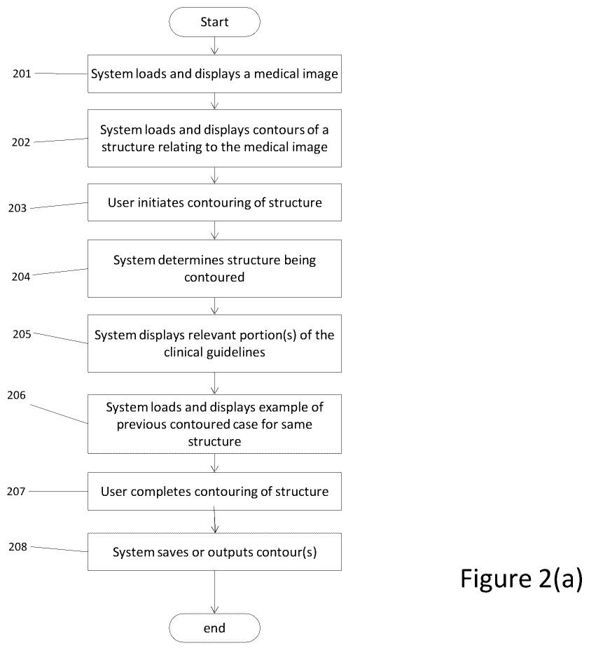

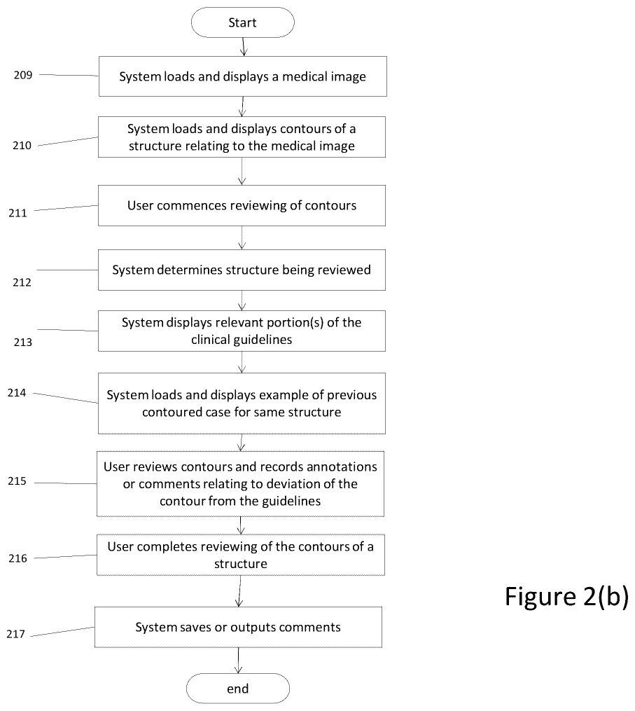

In an alternative embodiment of the invention, as illustrated in FIG. 2(a), the method proceeds in a largely similar manner. In this embodiment of the invention one or more pre-existing contours on a medical image can be edited or reviewed, or one or more new contours can be added to a medical image that already has pre-existing contours. This is in contrast to the previous embodiment as described in FIG. 1, where the initial medical image does not have any contours, and the contours are generated by the system user. The system loads at least one a medical image (which may be 2D or 3D, or a time-series of medical images), at step 201. As for the FIG. 1 embodiment, the medical image is preferably a CT image but maybe a different imaging methodology such as MRI, PET, SPECT or Ultrasound. The system also loads and displays one or more pre-existing contours or delineations on anatomical structures visible in the medical image, as indicated at step 202, before any further contouring is performed. The one or more pre-existing contours can then be reviewed for compliance with the guidance for that specific contour, and/or edited to better conform to the guidance. These existing one or more delineations may have come from manual contours from a prior contouring session performed by a user (whether the same user or a different users), or from an auto-contouring system. The user then initiates contouring or review of the contour of the medical image at step 203, and the system and method determines that the user has initiated contouring on the medical image. In an embodiment of the invention determination that the user has initiated contouring or reviewing of a contour is determined when the user starts to draw or edit a contour of the structure on the at least one medical image. In an alternative embodiment of the invention, the determination that a pre-existing contour is to be edited or reviewed is done using eye-tracking hardware and/or software to identify the location of the users gaze on the at least one medical image.

This contouring of the determined structure on the medical image by the user may be manual, semi-automatic or automatic contour of the image with pre-existing contours. This contouring may be de-novo delineation of a new structure on the medical image with one or more existing contours, or the editing of one of the one or more pre-existing delineations already shown on the medical image. The system and method then determines which anatomical structure on the medical image is being contoured/or having a contour reviewed by the user at step 204. Preferably, this is done automatically by the system. On determining which structure in the medical image is being contoured, the system may display guidance information for the structure being contoured to the system user at step 205. Preferably, the guidance information is one or more of the relevant portions of guidelines to the user, preferably via the system's user interface. The user interface may be on the screen where the contouring is occurring, or on a separate screen that is another part of the user interface. On determining the structure on the medical image that is being contoured, the system may preferably load and display an atlas at step 206 showing an example contour of the structure that has been drawn according to guidelines. Steps 205 and 206 may both be performed or only one of these steps may be performed. Furthermore, the step 206 may be performed independently of step 205 being performed—thus step 206 may be an optional step within the workflow indicated in FIG. 2. As with the previously described workflow, once the user has completed the contouring, at step 207, the system can preferably store, save or output one or more of the contours created by the user for further use in the clinical workflow at step 208. In some embodiments of the invention, this stage of saving/storing outputting can be completed in stages as the contouring is performed, so that initially only a partial delineation of the structure being contoured is saved for further use, this continues as the contouring continues, until the completed contour is saved. Output of the contour would normally be in a format commonly used within the medical device community, such as DICOM format. DICOM is also an image transfer protocol, thus the data may be transferred to another devices such as a Treatment Planning System (TPS) or a Picture Archiving and Communications System (PACS). Alternatively, the contours (partial or complete) may be stored to a local or network file system. In an embodiment of the invention, the steps of the method for reviewing pre-existing contours as described above, are repeated so that multiple pre-existing contours on the medical image are reviewed.

In an alternative embodiment of the invention, as illustrated in FIG. 2(b), the method of the invention proceeds in a largely similar manner. In this embodiment of the invention one or more of the pre-existing contours on a medical image can be reviewed with respect to the guidance for the contouring of the structure on the medical image. The system loads at least one a medical image (which may be 2D or 3D, or a time-series of medical images), at step 209. As for the FIG. 1 embodiment, the medical image is preferably a CT image but maybe a different imaging methodology such as MRI, PET, SPECT or Ultrasound. The system also loads and displays one or more pre-existing contours or delineations on anatomical structures visible in the medical image, as indicated at step 210, before any review of the pre-existing contours is performed. These existing delineations on the medical image that are displayed to the user may have come from manual or semi-automatic contours from a prior contouring session performed by a user (whether the same user or a different users), or from an auto-contouring system. Preferably, the system displays the at least one medical image annotated with one or more pre-existing contours, where the pre-existing contours will be reviewed. It is then determined that the user has initiated reviewing at least one of the pre-existing contours of the medical image at step 211. In an embodiment of the invention, the determination that a pre-existing contour is to be edited or reviewed is done using eye-tracking hardware and/or software to identify the location of the users gaze on the at least one medical image.

The system and method determine that the user has initiated reviewing a pre-existing contour on the medical image, and then determine the anatomical structure that is associated with the pre-existing contour that is being reviewed on the medical image by the user at step 212. Preferably, this structure determination is done automatically by the system and method.

On determining which structure in the medical image that the pre-existing contour being reviewed relates to, the system may display guidance information for the contouring of that specific structure to the system user at step 213. Preferably, the guidance information is one or more of the relevant portions of guidelines to the user, preferably via the system's user interface. On determining the structure on the medical image that is being contoured, the system may preferably load and display an atlas at step 214 showing the structure drawn according to guidance information Steps 213 and 214 may both be performed or only one may be performed. The system then reviews one or more contours at step 215, and provides feedback on the contour. In an embodiment of the invention, the method may further comprise the step of completing the review of the pre-existing contour or editing the pre-existing contour in accordance with the displayed guidance.

Completing the review of the pre-existing contour may also comprise providing feedback on the contour. This review or feedback may take the form of annotations being added to the image or the contour, or comments written for the contour. Comments and annotations may be recorded within the same system, or separately in an independent system. As with the previously described workflow, once the user has completed the review of the pre-existing contour, at step 216, the system stores, saves or outputs the contour annotations or comments created by the user for further use in the clinical workflow at step 217. The method may also include the step of outputting the or the reviewed contour associated with the image. Output of such a review of contours would normally be in a format commonly used within the medical device community, such as DICOM Structured Report format, although proprietary formats may be used by such a system. Comments or reports on the contour generated by the user at step 216 may be recorded in document formats such as MS Word or PDF documents. This may be stored within oncology information systems. The review at 215 may also be performed orally with the user explaining or giving feedback on guideline deviations to another user.

In either the ab initio contouring method as described above, or the review of pre-existing contours, the determination that the user has initiated contouring or reviewing of a contour is determined when the user starts to draw or edit a contour of the structure on the at least one medical image.

The steps of detecting the correct structure and linking this to the guidance for contouring and/or contour reviewing are core to this invention.

The structure in the medical image that is being contoured, or that has a pre-existing contour that is being reviewed by the user may be determined in many ways. Preferably the structure will be determined automatically. The examples given below are provided as possible embodiments of the invention but should not be considered exhaustive.

In an embodiment of the invention, the contouring system determines the structure being contoured or reviewed using a contour name of the structure being contoured. Methods to determine the structure being contoured include;

Where the User Interface prompts the user to specify a contour name for the contour currently being reviewed or generated, a look-up table or database can be used to relate user specified structure names back to standard naming conventions (e.g. [8]) thus the structure that is being contoured is indicated by the user. Similarly, where auto-contouring of a structure on a medical image is used as a starting point for contouring, the name of the structure will be specified by the auto-contouring system, and a similar look-up table can be applied.

In the different embodiments of the invention, the determination of the structure being reviewed or contoured may be made by finding a mapping between the at least one medical image and at least one atlas image. In the various embodiments of the invention, the mapping is done using image registration. Preferably, image registration [9] techniques can be used to find a mapping between an atlas image and the patient medical image. Image registration is the process by which an alignment, or spatial correspondence, also refer to as a mapping between two or more images is determined. One or more of rigid, affine and deformable image registration methods may be used to correct for differences within images to be aligned to various extents. In rigid alignment, translation and rotation of the images may be performed. In affine alignment, shearing and scaling of the images may be performed in addition to translation and rotation. In deformable alignment, translation of individual points within an image is able to be performed. Using this mapping and as drawing of the contour progresses, the location of the medical image at which the user is contouring on the patient medical image is mapped to the corresponding location on the atlas image. The spatial correspondence can be used to determine the structure in the patient medical image that is being contoured, by finding in the existing set of structures defined on the atlas image, the closest structure to the contour being drawn on the patient medical image., The notion of closest structure can be quantified for example using a scoring system based on contour similarity measures between the user drawn contour and the atlas set of contours. For example, the Dice Similarity Coefficient measuring the spatial overlap between the drawn contour and the set of atlas contours can be used to estimate the closest structure with the maximum overlap. A measure such as the Average Surface Distance between the drawn contour and the set of atlas contours can be used to estimate the closest structure with the minimum spatial distance on average. The closest structures can also be selected by comparing the centroid of two contours or any combination in a voting scheme of contour or shape similarity measures [10]. Note that the user contour may not be complete, thus the similarly comparison may be performed on a partial region of the full drawn contour or atlas contour.

In a preferred embodiment of the invention, either for contour generation, or for contour review, Machine Learning may be used to predict the structure that is being contoured, or the contour that is being reviewed. Preferably, the determination of the structure being reviewed or contoured is made by comparison to contours produced using Machine Learning based auto-contouring. Machine learning based methods can preferably be used to determine which organ is being contoured or having a contour to be reviewed, from the location in the image at which the user is contouring/reviewing [11]. One such method is to apply machine learning based auto-contouring to the image (with or without displaying this result) as a way of determining the similarity or proximity of the user drawn contour to the auto-contour. Alternatively, the appearance of the image where the user is drawing can be mapped to directly to classification of which organ is being contoured using machine learning methods. For example, information of a given image patch from the medical image together with information on which structure that image patch relates to be used to train a classifier, to predict the structure for the given image patch. This classifier may then be used to predict the structure given a patch of the medical image around the location on the medical image where the user is contouring.

A similar approach may be taken by using a “dictionary” of structure image appearances. The patch around the location on the medical image where the user is contouring, or is reviewing an existing contour is compared using image similarity measures (such as those normally used for image registration) to each patch within the atlas image. Each patch within the atlas image is assigned the structure(s) which it may represent. The structure on the medical image that is being contoured, or where a pre-existing contour is being reviewed by the user may be determined by finding which atlas image patch is most similar to the patch around the location on the medical image where the user is contouring or reviewing a pre-existing contour, and thus assigning the structure(s) from the atlas. Where multiple structures could be assigned to a patch, comparison of multiple patches may be used to determine the most likely candidate structure.

Alternatively, a partially contoured structure, that is being contoured by the user, could be used with a machine learning classifier to determine the structure on the medical image being contoured. For example, a set of partial contours together with information on which structure they will become when completed may be used to train a classifier, to predict the structure for a given partial structure. This classifier may then be used to predict the structure given a partial user contour of the structure.

Various machine learning methods, known to those skilled-in-the-art and that may be used for machine learning based auto contouring may include but are not limited to; convolutional neural networks, random-forest methods, and support vector machines.

The relative location and proximity of the partially drawn user contour to the other previously identified structures on the medical image can be used, in association with demographic data from previous patient cases, to identify the structure being contoured on the medical image. For example, the heart is known to exist between the two lungs, and above the liver. Therefore, if the user has previously contoured the lungs and liver on the medical image and is now contouring on the medical image between the lungs and above the liver as shown on the medical image, the system can identify the new structure on the medical image as the heart.

Such approaches to determine the structure on a medical image being contoured or reviewed may be applied individually or in combination. The determination of the structure on the medical image may be done automatically. A ranking of probable structures on the medical image may also be created, such that the structure that is determined to be the most likely structure being reviewed or contoured by the user is the first ranked. The guidelines or atlases for this first ranked structure can then be displayed to the user, preferably automatically. A simple interface can be provided to the user to dismiss this structure if it is not what they are considering and the guidelines or atlas for the next most likely candidate can then be displayed.

On determining the structure in the medical image that is being contoured, or has a pre-existing contour that is being reviewed, the appropriate guidance (preferably guideline and/or atlas) is displayed to the user via the user interface, or some other display means. A database may be used to store the relationship between the identified structure and the guidance information. This database can either directly store the guidance information (such as guideline data) for retrieval or be used to store the file and location of information within the file such that the system can retrieve the appropriate file and display the guidance information for the corresponding structure. Such a database could be pre-configured, configured by the user or system administrator, or automatically configured from a remote service.

Mapping of the guidance (guideline and atlas) information into such a database could be performed manually as an initial configuration, or automatically populated using natural language processing of the published guidelines to detect the relevant passages. Such natural language processing can consider the anatomical names used in the guidelines, together with analysis of formatting, such as headings, to determine the structure being discussed—as opposed to occurrences of a structure being referred to as defining the contouring of another structure.

Image registration between the medical image being reviewed or contoured and the atlas image can be computed. This the image registration can be used to determine the equivalent location within the atlas image as the user navigates in the medical image. For example, the location of the user's “brush” in the medical image, for use during the contouring can be mapped via the image registration to the atlas image. The atlas image and contour can then be displayed through this location on the atlas image. This enables the user to interactively see the appropriate image slices of the atlas image as they contour.

It is still helpful for the guidance (guidelines and/or atlas) to be displayed to the user in a situation where previously generated contours are being reviewed by the user for consideration for editing, but the user has not commenced editing of the pre-existing contours. In this instance no physical input to the system, such as the motion of the “brush” tool for contouring, by the user can be used to determine what contour the user wishes to consider and what guidelines would be appropriate to show. In such a scenario the structure of interest can be determined, preferably automatically by using eye-tracking hardware and/or software connected to the system to identify the location of the users gaze on the at least one medical image. Where the users gaze is on the patient image, the location of their gaze together with the methods described above, can be used to determine the structure under consideration, and the relevant guidance (guidelines and/or atlas) can be displayed to the user via the system user-interface.

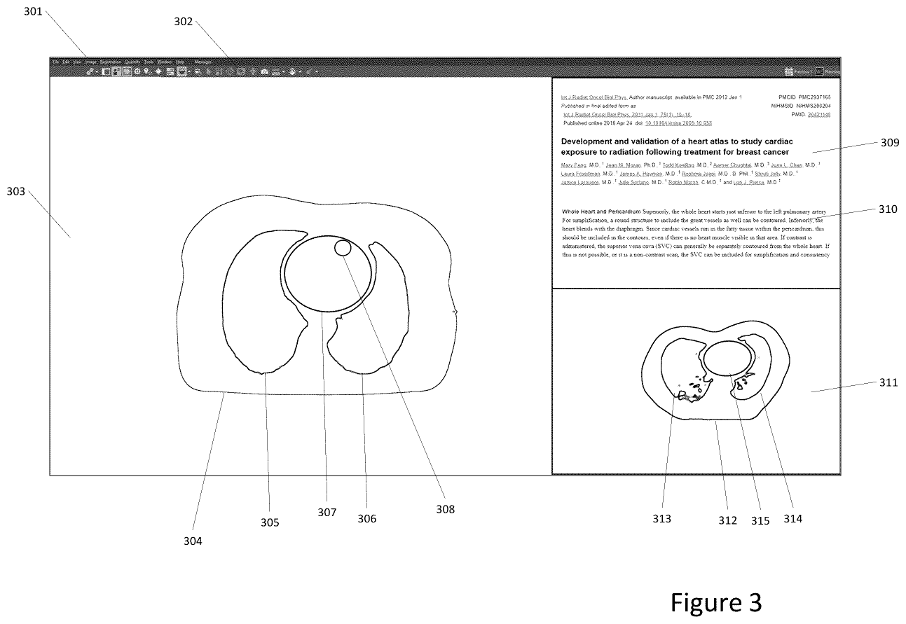

An example user-interface is illustrated in FIG. 3. Standard application menu items are shown at 301, with an application toolbar shown at 302. Such items would be expected to be present in such a system but are not essential to the invention. Menus and toolbars may be used by the user to change interaction mode between visualisation and drawing. The patient medical image is shown in visualisation pane 303. In this stylized example a number of structures are shown by outline; the patient skin—304, the right lung—305, the left lung—306, and the heart—307. The small circle indicated as 308, represents the user's cursor location as if the user was in the process of delineating the heart. In the diagram, the system has determined the user is contouring the heart on the medical image and is displaying the appropriate guidance being followed at 309, together with the specific guidance for contouring the heart at 310. The user-interface is also displaying an atlas image appropriate to the patient and identified anatomical structure, the heart (at 315), in pane 311. The atlas image shows the patient at 312, the right lung at 313, the left lung at 314 and the heart at 315. Having detected the user is contouring the heart, the atlas would display a contour for the heart drawn according to the guidelines. As shown, the interface is on a single screen, but it will also work over different split screens.

Where the system is displaying an atlas to the user of the appropriate structure, and the patient and atlas images are 3D medical images, it is also helpful that the atlas are displaying the same slice location. This can be achieved by linking the location in the patient image, as indicated by the user's mouse or the use of eye tracking hardware or software, to the navigation of the location in the atlas using deformable image registration to calculate the spatial correspondence between the two. Furthermore, the view angle with respect to the patient axis can be linked between the atlas and the patient to ensure the same cross-section through the patient is displayed.

Thus, the disclosed invention solves the problem of ensuring that a clinician contouring an anatomical structure on a medical image, or reviewing pre-existing contours on a medical image has the relevant guidance (guideline or atlas) information easily accessible for that structure during the process of contouring or contour reviewing.
[8] Mayo CS, Moran JM, Bosch W, Xiao Y, McNutt T, Popple R, Michalski J, Feng M, Marks LB, Fuller CD, Yorke E. American Association of Physicists in Medicine Task Group 263:standardizing nomenclatures in radiation oncology. International Journal of Radiation Oncology* Biology* Physics. 2018 Mar 15; 100(4):1057-66.
[9] Maintz JA, Viergever MA. A survey of medical image registration. Medical image analysis. 1998 Mar 1; 2(1):1-36.
[10] Veltkamp, Remco. (2001). Shape matching: Similarity measures and algorithms. Proceedings-International Conference on Shape Modeling and Applications, SMI 2001. 188-197. 10.1109/SMA.2001.923389.
[11] Yang J, Veeraraghavan H, Armato III SG, Farahani K, Kirby JS, Kalpathy-Kramer J, van Elmpt W, Dekker A, Han X, Feng X, Aljabar P. Autosegmentation for thoracic radiation treatment planning: a grand challenge at AAPM 2017. Medical physics. 2018 Oct; 45(10):4568-81.

Examples of this invention may be applied to any or all of the following: Picture archiving and communication systems (PACS); Advanced visualisation workstations; Imaging Acquisition Workstations; Web-based or cloud-based medical information and image systems; Radiotherapy Treatment planning system (TPS); Radiotherapy linear accelerator consoles; Radiotherapy proton beam console.

The present invention has been described with reference to the accompanying drawings. However, it will be appreciated that the present invention is not limited to the specific examples herein described and as illustrated in the accompanying drawings. Furthermore, because the illustrated embodiments of the present invention may for the most part, be implemented using electronic components and circuits known to those skilled in the art, details will not be explained in any greater extent than that considered necessary as illustrated above, for the understanding and appreciation of the underlying concepts of the present invention and in order not to obfuscate or distract from the teachings of the present invention.

The invention may be implemented in a computer program for running on a computer system, at least including code portions for performing steps of a method according to the invention when run on a programmable apparatus, such as a computer system or enabling a programmable apparatus to perform functions of a device or system according to the invention.

A computer program is a list of instructions such as a particular application program and/or an operating system. The computer program may for instance include one or more of: a subroutine, a function, a procedure, an object method, an object implementation, an executable application, an applet, a servlet, a source code, an object code, a shared library/dynamic load library and/or other sequence of instructions designed for execution on a computer system. Therefore, some examples describe a non-transitory computer program product having executable program code stored therein for automated contouring of cone-beam CT images.

The computer program may be stored internally on a tangible and non-transitory computer readable storage medium or transmitted to the computer system via a computer readable transmission medium. All or some of the computer program may be provided on computer readable media permanently, removably or remotely coupled to an information processing system. The tangible and non-transitory computer readable media may include, for example and without limitation, any number of the following: magnetic storage media including disk and tape storage media; optical storage media such as compact disk media (e.g., CD ROM, CD R, etc.) and digital video disk storage media; non-volatile memory storage media including semiconductor-based memory units such as FLASH memory, EEPROM, EPROM, ROM; ferromagnetic digital memories; MRAM; volatile storage media including registers, buffers or caches, main memory, RAM, etc.

A computer process typically includes an executing (running) program or portion of a program, current program values and state information, and the resources used by the operating system to manage the execution of the process. An operating system (OS) is the software that manages the sharing of the resources of a computer and provides programmers with an interface used to access those resources. An operating system processes system data and user input, and responds by allocating and managing tasks and internal system resources as a service to users and programs of the system.

The computer system may for instance include at least one processing unit, associated memory and a number of input/output (I/O) devices. When executing the computer program, the computer system processes information according to the computer program and produces resultant output information via I/O devices.

In the foregoing specification, the invention has been described with reference to specific examples of embodiments of the invention. It will, however, be evident that various modifications and changes may be made therein without departing from the scope of the invention as set forth in the appended claims and that the claims are not limited to the specific examples described above.

Those skilled in the art will recognize that the boundaries between logic blocks are merely illustrative and that alternative embodiments may merge logic blocks or circuit elements or impose an alternate decomposition of functionality upon various logic blocks or circuit elements. Thus, it is to be understood that the architectures depicted herein are merely exemplary, and that in fact many other architectures can be implemented which achieve the same functionality.

Any arrangement of components to achieve the same functionality is effectively ‘associated’ such that the desired functionality is achieved. Hence, any two components herein combined to achieve a particular functionality can be seen as ‘associated with’ each other such that the desired functionality is achieved, irrespective of architectures or intermediary components. Likewise, any two components so associated can also be viewed as being ‘operably connected,’ or ‘operably coupled,’ to each other to achieve the desired functionality.

Furthermore, those skilled in the art will recognize that boundaries between the above described operations merely illustrative. The multiple operations may be combined into a single operation, a single operation may be distributed in additional operations and operations may be executed at least partially overlapping in time. Moreover, alternative embodiments may include multiple instances of a particular operation, and the order of operations may be altered in various other embodiments.

However, other modifications, variations and alternatives are also possible. The specifications and drawings are, accordingly, to be regarded in an illustrative rather than in a restrictive sense.

In the claims, any reference signs placed between parentheses shall not be construed as limiting the claim. The word ‘comprising’ does not exclude the presence of other elements or steps then those listed in a claim. Furthermore, the terms ‘a’ or ‘an,’ as used herein, are defined as one or more than one. Also, the use of introductory phrases such as ‘at least one’ and ‘one or more’ in the claims should not be construed to imply that the introduction of another claim element by the indefinite articles ‘a’ or ‘an’ limits any particular claim containing such introduced claim element to inventions containing only one such element, even when the same claim includes the introductory phrases ‘one or more’ or ‘at least one’ and indefinite articles such as ‘a’ or ‘an.’ The same holds true for the use of definite articles. Unless stated otherwise, terms such as ‘first’ and ‘second’ are used to arbitrarily distinguish between the elements such terms describe. Thus, these terms are not necessarily intended to indicate temporal or other prioritization of such elements. The mere fact that certain measures are recited in mutually different claims does not indicate that a combination of these measures cannot be used to advantage.

1-26. (canceled)

27. A method for contouring a medical image in a contouring system comprising the steps of:
providing at least one medical image to be contoured by a user;
determining that the user has initiated contouring of a structure on the medical image;
the contouring system determining the structure that is being contoured;
in response to the determination of the structure being contoured the system displaying guidance for the contouring of the determined structure;
contouring the determined structure in accordance with the displayed guidance.

28. A method for reviewing contours on a contoured medical image comprising the steps of:
providing at least one medical image annotated with one or more pre-existing contours;
displaying the at least one medical image annotated with one or more pre-existing contours;
determining that the user has initiated reviewing a pre-existing contour on the medical image;
determine the structure associated with the pre-existing contour being reviewed by the user;
in response to the determination of the structure displaying guidance for the contouring of the determined structure.

29. A method as claimed in claim 28, further comprising the step of completing the review of the pre-existing contour or editing the pre-existing contour in accordance with the displayed guidance.

30. A method as claimed in claim 29, wherein completing the review of the pre-existing contour comprises providing feedback on the contour.

31. A method as claimed in claim 30, wherein the feedback is provided as comments or annotations on the contour.

32. A method as claimed in claim 27, further comprising the step of outputting the user-initiated contour.

33. A method as claimed in claim 27, wherein the determination that the user has initiated contouring or reviewing of a contour is determined when the user starts to draw or edit a contour of the structure on the at least one medical image.

34. A method as claimed in claim 28, wherein the determination that a pre-existing contour is to be edited or reviewed is done using eye-tracking hardware and/or software to identify the location of the users gaze on the at least one medical image.

35. A method according to claim 27, wherein the guidance for contouring is at least one of: clinical guidelines for contouring the determined structure or at least one atlas image showing at least one example contour for the determined structure.

36. A method as claimed in claim 27, wherein the determination of the structure being reviewed or contoured is made by finding a mapping between the at least one medical image and at least one atlas image; wherein the mapping is done using image registration; wherein the image registration comprises one or more of: deformable registration, affine registration or rigid registration.

37. A method as claimed in claim 27, further comprising using Machine Learning to predict the structure being contoured.

38. A method as claimed in claim 37, wherein the determination of the structure being reviewed or contoured is made by comparison to contours produced using Machine Learning based auto-contouring.

39. A method as claimed in claim 27, wherein the determination of the structure being reviewed or contoured on the medical image is done using eye-tracking hardware and/or software to identify the location of the users gaze on the at least one medical image.

40. A method as claimed in claim 27, wherein the contouring of the determined structure by the user is one of manual, semi-automatic or automatic contouring.

41. A method as claimed in claim 40, wherein the contouring system determines the structure being contoured or reviewed using a contour name of the structure being contoured; wherein the contour name is provided to a look-up table so the name is related to a standardised naming convention.

42. A method as claimed in claim 27, wherein the steps of the method are repeated so that multiple structures in the at least one medical image are contoured.

43. A method as claimed in claim 28, wherein the pre-existing contours displayed to the user are either manual contours from a previous contouring session, or automatically produced contours.

44. A method as claimed in claim 28, wherein the steps of the method are repeated so that multiple pre-existing contours on the medical image are reviewed.

45. A method as claimed in claim 27, wherein the at least one medical image is one a CT scan, an MRI scan, a PET scan, a SPECT scan or an ultrasound scan.

46. A system for contouring of at least one medical image comprising:
a display for displaying at least one medical image to be contoured by a user;
a processor for determining that the user has initiated contouring of a structure on the medical image;
the processor determining the structure that is being contoured; and
in response to the determination of the structure the system displaying guidance to the user for the contouring of the determined structure; so that the determined structure can be contoured in accordance with the displayed guidance.
